(19) United States
(12) Patent Application Publication
(10) Pub. No.: US 2005/0180965 A1
(43) Pub. Date: Aug. 18, 2005

(54) Heat-reversible composition treating hyposalia and asialia

(76) Inventor: Jack Auzerie, Saint-Eulalie (FR)

Correspondence Address: YOUNG & THOMPSON, 745 SOUTH 23RD STREET, 2ND FLOOR, ARLINGTON, VA, 22202, US

(21) Appl. No.: 10/505,606

(22) PCT Filed: Mar. 26, 2003
(86) PCT No.: PCT/FR03/00949

(30) Foreign Application Priority Data
Mar. 26, 2002 (FR) 02/03728

(51) Int. Cl. A61K 38/47; A61K 9/14

(52) U.S. Cl. 424/94.61; 424/486; 424/488

(57) ABSTRACT

A heat-reversible composition designed to compensate salivary deficiencies, includes at least a polymeric compound whose viscosity is low at room temperature and which increases at oral temperature so as to reach optimal viscosity at oral temperature to gelling point.

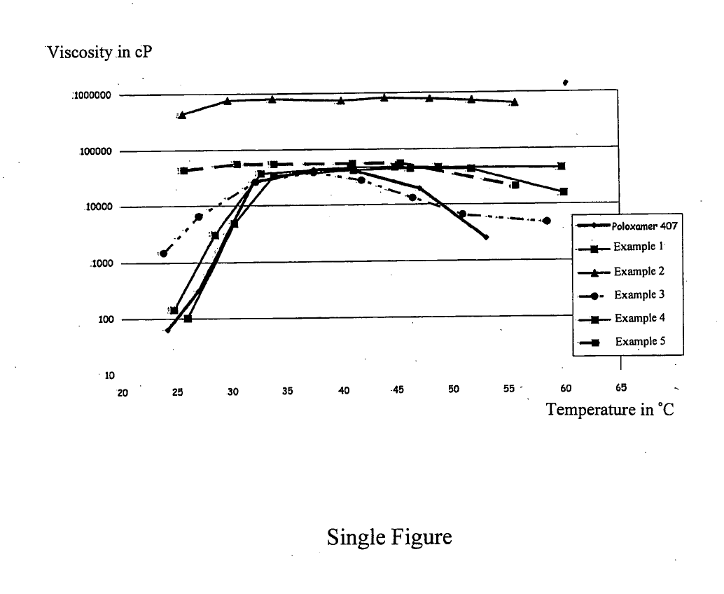

The present invention covers a pharmaceutical composition of artificial saliva, permitting compensating insufficiencies of salivary secretion called xerostomia, which manifests as a decrease in salivary secretion called hyposialia or total absence of secretion called asialia.

Xerostomia manifests itself by dryness of the mouth. The patient suffers within his mouth and on his tongue burning, pricking, his mouth seems pasty and sticky. Patients suffering from this malady have difficulty in swallowing and eating particularly dry foods. In certain cases, the mere taking of tablets and capsules is difficult.

Food tastes insipid, without flavor and even speech is difficult.

Over the long term, xerostomia can give rise to lesions of the ulceration type of the buccal mucosa, of the tongue and of the lips.

Moreover, the absence of saliva has consequences on buccodental hygiene and there is noted in patients an increase in the number of cavities, the appearance of buccal mycosa particularly difficult to eradicate.

The absence of saliva gives rise to sleep difficulties with the direct consequence of physical fatigue.

The causes of xerostomia are numerous and particularly of origin from medication.

There can be cited numerous families of medication that are very diverse, but among the most used can be cited those for anorexia, anxiety, anticholinergesis and antispasmodics, anticonvulsants, antidepressants, antidiarrheals, antiemetics, antihistamines, antihypertensives, certain analgesia and anti-inflammatories, antiparkinsons, antipsychotics, broncho-dilators, decongestants, diuretics, sedatives and muscular relaxants.

Scorgen malady, auto-immune malady, the type of arthritis which hits women after menopause, decreases the secretion of the lacrymal glands but also the salivary glands.

In certain cases, sick persons infected with the HIV virus and more particularly in infants, the swelling of the salivary glands give rise to xerostomia.

There can be cited other afflictions whose effects give rise to xerostomia: rheumatoid arthritis, erythematic lupus, diabetes, hypertension, endocrinal disorders, or thyroidal dysfunctions.

In certain cases of cancer, according to the location thereof, radiotherapy used to eradicate it can destroy the salivary glands irreversibly, by fibrosis of the salivary glands. There can be rapid episodes which follow radiotherapy treatments, but xerostomia is then only passing.

The composition according to the present invention has the object of overcoming the consequences connected with xerostomia, given that the known treatments are not satisfactory.

The prior art proposes medications which act systemically.

Pilocarpine is an active substance with cholinergenic effect which is used to stimulate the muscles disposed about the salivary glands to increase the production of saliva. Cevimeline also stimulates the production of saliva.

Nevertheless, the secondary effects are great because the principles act also at other points and give rise to the appearance of sweating, gastrointestinal disorders, hypotension, rhinitis, visual troubles.

Contraindications are for patients suffering from asthma, glaucoma and pregnant women.

Moreover, when the salivary glands are totally destroyed, stimulation has no tangible effect.

Other products act locally to stimulate salivation in the buccal area, such as sugarless candy, chewing gum or maltose base formulations.

The effect is temporary and these products are not suitable to deal with serious maladies of the salivary glands.

Products have been proposed as a substitute for saliva, such as mouthwashes based on chlorhexidine or hexamedine. Such products have only a small residual effect, requiring repeated application.

European patent application EP 788 368 discloses aqueous preparations of polymers. The commercial product is called Xialine, in spray form.

Nevertheless, it is difficult to obtain regular impregnation of the buccal mucosa because of the viscosity of the preparation, which is substantially constant no matter what the temperature.

There is also known from patent application WO96 34608, a composition having a lubricating function, particularly in the case of xerostomia. This application provides a composition which includes a β-glucane polymer so as to give to it a rheological behavior near that of saliva.

There is thus sought a viscosity substantially identical to that of saliva. But this does not provide a composition very fluid at room temperature so as to facilitate application, whose viscosity increases to let it gel and permit a certain adherence on the buccal wall to prolong its action.

U.S. Pat. No. 5,496,541 discloses a dental product which uses a ternary surfactant system including a poloxamer combined with polysaccharides and cellulose. This composition permits obtaining a composition which adheres well to the mucosa and to the teeth.

Such a product does not have a viscosity which varies in the desired direction, namely, low viscosity at ambient temperature and an increase of viscosity at the buccal temperature, even achieving gelification.

The present invention provides a composition usable as a substitute for saliva, in a thermoreversible form.

This form permits compensating the drawbacks of the known compositions and particularly leads to a better impregnation of the buccal cavity because of the liquid and low viscosity nature during application. Gelification then permits good adherence to the buccal mucosa.

Other advantages will become apparent and particularly the possibility of marketing the composition in pressurized cans, in sterile form. This is beneficial for patients that have mucosal lesions.

The composition according to the present invention will now be described in detail as to different embodiments.

The composition according to the invention has as its first component a thermoreversible polymer selected from synthetic copolymers formed by ethylene oxide and propylene oxide, called Poloxamers, particularly Poloxamer 407, sold under the name Lutrol F 127.

This polymer permits keeping the product at ambient temperature in liquid form, whilst it becomes much more viscous at the temperature of the human body.

A second compound associated with the first is a bioadhesive polymer selected from the group of polyvinylpyrrolidone, caboxymethylcellulose and its salts, hydroxypropylmethylcellulose, hydroxypropylcellulose, alginates, derivatives of adragante and karaya gum arabic, alginic acid and its salts, polymers of acrylic acids, methacrylates and their derivatives.

Not necessarily but desirably, particularly to avoid microbial contamination, there can be added preservatives such as parahydroxybenzoic acid esters, sorbic acid, benzoic acid, quaternary ammonium salts such as benzalkonium chloride or chlorhexidine.

As a supplement, there could be added to this composition antioxidant substances so as to stabilize the characteristics of the product, particularly to avoid the phenomena of oxidation of the poloxameric derivatives.

As antioxidants, can be cited tocopherols, derivatives of vitamin E, ascorbyl palmitate, vitamin C, flavonoids of natural origin, isoflavonoids or polyphenols.

So as to approach the characteristics of saliva, there can be introduced enzymes present in saliva and which part in the digestion and buccodental hygiene, such as alpha-amylases, lysozyme and lactoferrin.

So as to illustrate the composition according to the invention, examples will now be given in a non-limiting but simply illustrative way.

### EXAMPLE 1

The quantities are expressed in % by weight.

| | |
|---|---|
| Poloxamer 407: | 15% |
| Hydroxypropylcellulose: | 1% |
| (Klucel EF) | |
| Purified water: | Quantity Sufficient |
| | to Make Up |
| | 100% |

To produce this composition, the operative mode consists in dissolving in purified water at ambient temperature the Poloxamer 407 with the help of a defloculator and in progressively adding the hydroxypropylcellulose.

The artificial saliva is agitated at 4° C. for several hours to ensure debubbling.

### EXAMPLE 2

| | |
|---|---|
| Poloxamer 407: | 15% |
| Hydroxypropylcellulose: | 0.5% |
| (Natrosol 250) | |
| Purified water: | Quantity Sufficient |
| | to Make Up |
| | 100% |

To make this composition, there is dissolved in purified water with agitation at ambient temperature the hydroxypropylcellulose.

There is then added the thermoreversible polymer progressively for defloculation.

The artificial saliva is agitated at 4° C. for several hours to ensure debubbling.

### EXAMPLE 3

| | |
|---|---|
| Poloxamer 407: | 15% |
| Karaya gum: | 0.5% |
| Purified water: | Quantity Sufficient |
| | to Make Up |
| | 100% |

To make this composition, there is dissolved the karaya gum in purified water with agitation at ambient temperature.

There is then added the thermoreversible polymer progressively for defloculation.

The artificial saliva is agitated at 4° C. for several hours to ensure debubbling.

### EXAMPLE 4

| | |
|---|---|
| Poloxamer 407: | 15% |
| Hydroxypropylmethylcellulose: | 2% |
| (Pharmacoat 606) | |
| Purified water: | Quantity Sufficient |
| | to Make Up |
| | 100% |

There are simultaneously dissolved the hydroxypropylmethylcellulose and the polymer in purified water with agitation at ambient temperature.

The artificial saliva is agitated at 4° C. for several hours to ensure debubbling.

### EXAMPLE 5

| | |
|---|---|
| Poloxamer 407: | 15% |
| Carbomers: | 0.5% |
| (Carbopol 5984) | |
| Purified water: | Quantity Sufficient |
| | to Make Up |
| | 100% |

The carbomer is first dissolved in purified water with agitation at ambient temperature.

The poloxamer is added progressively with defloculation.

The artificial saliva is agitated at 4° C. for several hours to ensure debubbling.

### EXAMPLE 6

| | |
|---|---|
| Poloxamer 407: | 15% |
| Hydroxypropylmethylcellulose: | 2% |
| (Pharmacoat 606) | |
| Vitamin E: | 2% |
| Purified water: | Quantity Sufficient |
| | to Make Up |
| | 100% |

The Poloxamer and the hydroxypropylmethylcellulose are dissolved simultaneously in purified water under agitation at ambient temperature with the introduction of the vitamin E.

The artificial saliva is agitated at 4° C. for several hours to ensure debubbling.

So as to show the effect of synergy obtained with the composition according to the present invention, the viscosity is measured with the help of a commercial apparatus.

There is seen on the attached single figure, that for the compositions of Examples 1 to 5, the viscosities all have a maximum of viscosity between 35 and 40° C., which corresponds to a gelification threshold in the desired region.

The viscosity is low at ambient temperature and increases to the buccal temperature. The polymeric composition with increasing viscosity has an optimum viscosity at the buccal temperature reaching the point of gelification.

This composition comprises a combination of a bioadhesive polymer with the polymer compound of increasing viscosity. This polymer is introduced into the composition in the amount of 1% to 30% by weight and the bioadhesive polymer is introduced into the composition in the amount of 0.1 to 10.0% by weight.

At ambient temperature, the composition is liquid, with a low viscosity, which permits its packaging in spray bottles, pressurized cans, in soft capsules, in flexible tubes or in sterile unit packagings.

1. Thermoreversible composition to compensate salivary insufficiency, characterized in that it comprises at least one polymeric compound whose viscosity is low at ambient temperature and which increases at the buccal temperature so as to reach an optimum viscosity at the buccal temperature up to the point of gelification.

2. Thermoreversible composition according to claim 1, characterized in that it comprises a bioadhesive polymer in combination with the polymeric compound of increasing viscosity.

3. Thermoreversible composition according to claim 2, characterized in that the bioadhesive polymer is selected from the family of polyvinylpyrrolidone, carboxymethylcellulose and its salts, hydroxypropylmethylcellulose, hydroxypropylcellulose, alginates, derivatives of adragante and karaya gum arabic, alginic acid and its salts, polymers of acrylic acids, methacrylates and their derivatives.

4. Thermoreversible composition according to claim 1, characterized in that it comprises an antioxidant.

5. Thermoreversible composition according to claim 1, characterized in that it comprises at least one enzyme such as alpha-amylases, lysozyme and lactoferrine.

6. Thermoreversible composition according to claim 1, characterized in that the polymer with increasing viscosity is introduced into said composition in the amount of 1 to 30% by weight and the bioadhesive polymer is introduced into said composition in the amount of 0.1% to 10.0% by weight.
